(19) United States
(12) Patent Application Publication
(10) Pub. No.: US 2021/0063395 A1
(43) Pub. Date: Mar. 4, 2021

(54) SENSOR FOR DETECTING FOOD-BORNE GASTROINTESTINAL IRRITANTS, CONTAMINANTS, ALLERGENS AND PATHOGENS

(71) Applicants: Orly YADID-PECHT, Calgary (CA); Raymond Joseph TURNER, Calgary (CA); Nikhil Suresh VASTAREY, Calgary (CA); Varun VIJ, Calgary (CA)

(72) Inventors: Orly YADID-PECHT, Calgary (CA); Raymond Joseph TURNER, Calgary (CA); Nikhil Suresh VASTAREY, Calgary (CA); Varun VIJ, Calgary (CA)

(21) Appl. No.: 17/054,394

(22) PCT Filed: May 10, 2019
(86) PCT No.: PCT/CA2019/050631

§ 371 Date: Nov. 10, 2020

Related U.S. Application Data
(60) Provisional application No. 62/669,758, filed on May 10, 2018.

(51) Int. Cl. G01N 33/569; G01N 21/76

(52) U.S. Cl. CPC G01N 33/56911; G01N 21/763

(57) ABSTRACT

Embodiments of the present disclosure relate to a sensor that alters its photoluminescent properties upon a binding event between a food-borne analyte and an analyte-specific aptamer (ASA). The ASA may recognize and bind the food-borne analyte, which is referred to herein as the binding event. In some embodiments of the present disclosure the ASA is a strand of single-stranded DNA (ssDNA). Some embodiments of the present disclosure the ASA may be conjugated with a chemically modified photoluminescent matrix material. In some embodiments of the present disclosure, the food-borne analyte may be conjugated with a quencher that may be incorporated into the system for reducing false signals.

### TECHNICAL FIELD

This disclosure generally relates to an apparatus and method for detecting food-borne analytes such as gastrointestinal irritants, contaminants, allergens and pathogens. Some embodiments of this disclosure relate to a sensor for detecting food-borne analytes in samples, including in samples of food.

### BACKGROUND

Food-borne gastrointestinal irritants, contaminants, allergens and pathogens are increasingly of concern for stakeholders in the food industry, including but not limited to growers, producers, processers, distributors, retailers and consumers.

Gluten is but one example of a food-borne gastrointestinal irritant, contaminant, allergen or pathogen that can cause negative health repercussions when consumed by subject with a food-borne sensitivity. Gluten is a term used to describe various proteins that are found within wheat and other grains. Gluten-containing grains can cause wheat allergy (WA) or celiac disease (CD). Gluten comprises at least two constituent proteins that are toxic to a portion of the general population. The toxic constituent-proteins are gliadin and glutenin. Typically, gliadin is present as peptide monomers and glutenin is an aggregated protein complex.

In both WA and CD the reaction to gluten is mediated by T-cell activation in the gastrointestinal mucosa. However, in WA it is a cross-linking of immunoglobulin E (IgE) by repeated sequences within the gluten peptides that triggers the release of chemical mediators, such as histamine, from immune cells such as basophils and mast cells. In contrast, CD is an autoimmune disorder that involves specific serologic-autoantibodies, most notably serum anti-tissue transglutaminase (tTG) and anti-endomysial antibodies (EMA).

Besides CD and WA, there are also reported cases of gluten reactions in which neither allergic nor autoimmune mechanisms are involved. These cases are generally defined as gluten sensitivity (GS).

Some strains of Escherichia coli (E. coli) E. coli are each another example of a food-borne gastrointestinal irritant, contaminant, allergen or pathogen that can cause negative health repercussions when consumed by subject. The common sources of exposure to these E. coli strains are raw or undercooked meat products, raw milk, and fecal contamination of vegetables. Exposure to these E. coli strains can cause abdominal cramps, fever, vomiting and diarrhea that may in some cases progress to hemorrhagic colitis. Most patients recover within 10 days, but in a small proportion of patients (particularly young children and the elderly), the exposure may result to a life-threatening disease, such as haemolytic uraemic syndrome (HUS). HS is characterized by acute renal failure, haemolytic anaemia and thrombocytopenia. It is estimated that up to 10% of patients with such E. coli infections may develop HUS, with a case-fatality rate ranging from 3 to 5%. Overall, HUS is the most common cause of acute renal failure in young children. Around 25% of HUS patients and chronic renal sequelae can have neurological complications such as seizure, stroke and coma, usually mild.

Therefore, the stakeholders in the food industry require a sensitive, cost-effective and fast way to detect the presence of food-borne gastrointestinal irritants, contaminants, allergens or pathogens in raw, cooked, manufactured, processed, stored and prepared food.

Known analytical methods for characterizing markers of gastrointestinal irritants, contaminants, allergens and pathogens include, but are not limited to: isoelectric focusing (IEF), matrix-assisted laser desorption ionization time-of-flight mass spectroscopy (MALDI-TOF-MS), polyacrylamide gel electrophoresis (PAGE), high performance resolution capillary electrophoresis (HPCE), reversed-phase high-performance liquid chromatography (RP-HPLC), size-exclusion HPLC (SE-HPLC), enzyme-linked immunosorbent assay (ELISA), immunoblotting, and polymerase chain reaction (PCR). Currently, detection of gluten proteins is typically based on: an immunological approach (mainly ELISA); a proteomic approach involving mass spectroscopy, which is not portable and too expensive for practical viability; or a genomic approach that involves PCR, which works on same concept as ELISA except that DNA is used instead of enzyme as a detector unit. Most of these approaches are incapable of providing reliable and highly sensitive detection of markers for gastrointestinal irritants, contaminants, allergens and pathogens. Of these approaches, the most sensitive and reliable approaches are ELISA and PCR, which are both complicated, and usually time consuming such that practical applicability is limited. Although there have been advancements in PCR in terms of shorter time spans and miniaturized thermocyclers, there has not been any portable commercial detection device for food allergens based on this technology.

The accurate and reliable detection of food-borne gastrointestinal irritants, contaminants, allergens and pathogens is difficult since many of these compounds comprise markers, such as proteins, that are soluble in alcohol and/or in dilute acids. The difficulties in accurately and reliably detecting food-borne gastrointestinal irritants, contaminants, allergens and pathogens in food may also be due to: (i) the complexity of the food matrix and the constituent proteins; (ii) the presence of several interfering proteins in the food matrix due to additives; (iii) the fact that processed food samples are very complex and depend upon a variety of extraction and preparation measures; (iv) a lack of readily available reference materials; and, (v) a lack of sensing techniques for the purpose of calibration or validation of detection results.

### SUMMARY

Some embodiments of the present disclosure relate to a photoluminescence based sensor that provides a suitable and easy approach for detecting food-borne gastrointestinal irritants, contaminants, allergens or pathogens in food. Collectively, the food-borne gastrointestinal irritants, allergens or pathogens may be referred to herein as food-borne analytes. Photoluminescence-based sensors are a low cost, fast and robust technology. Photoluminescence-based sensors of the present disclosure are also sensitive enough that they may detect binding events with the analyte of interest when the food-borne analyte of interest is present in the picomolar range.

Some embodiments of the present disclosure relate to a photoluminescent sensor for detecting the presence of a food-borne analyte. The sensor comprises a food-borne analyte-specific aptamer that can alter the photoluminescent properties of the sensor when a binding event occurs between the analyte-specific aptamer and the food-borne analyte. The sensor may be designed to detect a binding event with a wide variety of analytes. For example, the sensor may be designed to selectively detect one or more analytes of interest by including an aptamer sequence and one or more properties that are specific to the analyte(s) of interest.

### BRIEF DESCRIPTION OF THE DRAWINGS

These and other features of the present disclosure will become more apparent in the following detailed description in which reference is made to the appended drawings:

FIG. 1 is a schematic diagram of one embodiment of the present disclosure that relates to a photoluminescence-based sensor;

FIG. 2 is a schematic diagram that shows an example of fluorescence intensity vs. wavelength data that is expected of embodiments according to the present disclosure;

FIG. 3 shows examples of gluten-binding data and a ratio of fluorescent emissions of a Gli4-GO embodiment observed at 520 nm, wherein FIG. 3A shows an example of data obtained when the concentration of gliadin was between 0.1 ppm and 200 ppm; and, FIG. 3B shows an example of data that was obtained when the concentration of gliadin was between 0 to 20 ppm;

FIG. 4 shows examples of gluten-binding data and a ratio of fluorescent emissions of the Gli4-FAM-PEG-rGO/Q-glia embodiment when the concentration of gliadin was between 0 ppm and 27 ppm at observed at 520 nm;

FIG. 5 shows examples of binding data and a ratio of fluorescent emissions of a Gli4-FAM-PEG-rGO/Q-glia embodiment, wherein FIG. 5A shows data obtained when the concentration of lactose protein was between 0 ppm and 145 ppm in a buffer; and FIG. 5B shows data obtained when the concentration of peanut protein was between 0 ppm and 150 ppm in a buffer;

FIG. 6 shows examples of gliadin-binding data and a ratio of fluorescent emissions of a Gli4-FAM embodiment, wherein FIG. 6A shows an example of data that was obtained when the wider concentration range of gliadin lies between 0.1 ppm and 130 ppm, observed at 520 nm; and, FIG. 6B shows an example of data that was obtained when the concentration range of gliadin was smaller and lies between 0 to 10 ppm, observed at 520 nm;

FIG. 7 shows examples of ratio of fluorescent emissions of a Gli4-FAM-PEG-rGO/Q-glia embodiment observed at 520 nm when the concentration of gliadin-quencher hybrid was between 0 ppm and 57 ppm;

FIG. 8 shows examples of data that reflects a change in emission intensity of a Gli4-FAM-PEG-rGO/Q-glia embodiment in the presence of a competitor Q-glia when observed at 520 nm, wherein FIG. 8A shows the data obtained when the concentration of lactose protein was between 0 to 140 ppm; and, FIG. B shows the data obtained when the concentration of peanut protein was increased from 0 to 150 ppm;

FIG. 9 shows examples of change in fluorescence intensity data, wherein FIG. 9A shows the data obtained using a Gli4-FAM-PEG-rGO/Q-glia embodiment in the presence of about 13 ppm of standard gliadin when the concentration of peanut was increased from 0 ppm to 150 ppm; and FIG. 9B shows the data obtained using a Gli4-FAM-PEG-rGO/Q-glia embodiment in the presence of about 13 ppm of standard gliadin along with different concentrations of a competitor Q-glia (10, 20 and 50 ppm) when the concentration of peanut was increased from 0 ppm to 150 ppm;

FIG. 10 shows examples of data that was obtained by exposing embodiments of the present disclosure to various concentrations of gliadin in the absence of graphene oxide (GO);

FIG. 11 shows examples of spectral response data that was obtained by exposing embodiments of the present disclosure that included 10 μg/mL of GO to various concentrations of gliadin;

FIG. 12 shows examples of spectral response data that was obtained by exposing embodiments of the present disclosure that included 4 μg/mL of GO to various concentrations of gliadin;

FIG. 13 shows examples of spectral response data that was obtained by exposing embodiments of the present disclosure that included 2 μg/mL of GO to various concentrations of gliadin;

FIG. 14 shows examples of spectral response data obtained by exposing embodiment of present disclosure that includes 100 nM FAM-Aptamer immobilized-modified reduced GO to various concentrations of gliadin;

FIG. 15 shows examples of E. coli O157 binding data and a ratio of fluorescent emissions of the ESA1-FAM-PEG-rGO embodiment at different concentration of E. coli O157 observed at 520 nm; and

FIG. 16 shows examples of E. coli ATCC 25922 binding data and a ratio of fluorescent emissions of the ESA1-FAM-PEG-rGO embodiment at different concentration of E. coli ATCC 25922 observed at 520 nm.

### DETAILED DESCRIPTION

Embodiments of the present disclosure relate to a sensor that alters its photoluminescent properties upon a binding event between an analyte and an analyte-specific aptamer (ASA). Aptamers are single stranded nucleic acids or peptide molecules with a structure that can bind specifically with an analyte of interest. In some embodiments of the present disclosure the food-borne analyte may be a protein-based gastrointestinal irritant, contaminant, allergen or pathogen. Some non-limiting examples of food-borne analytes include: gluten, peanut, shellfish, soy, lactose, food-borne micro-organisms, such as Salmonella-type bacteria, Escherichia coli bacteria (E. coli), viruses, parasites, toxins and the like. The ASA may bind with a protein that is both a constituent of and specific to: gluten, peanut, shellfish, soy, lactose, food-borne micro-organisms, such as Salmonella-type bacteria, E. coli, viruses, parasites, toxins and the like or any combination thereof. The ASA may recognize and bind the analyte, or a constituent-protein of the analyte, which is referred to herein as a binding event. In some embodiments of the present disclosure the ASA is a strand of single-stranded DNA (ssDNA) that is specific to the analyte or a constituent-protein of the analyte. In one non-limiting example of the present disclosure, the food-borne analyte is gluten or a gluten constituent-protein and the ASA is a gluten-specific aptamer (GSA). The GSA may recognize and bind gluten or a gluten constituent-protein, in a binding event. In some embodiments of the present disclosure the GSA is a strand of single-stranded DNA (ssDNA) that is specific to the gluten constituent-protein, gliadin. In another non-limiting example of the present disclosure, the food-borne analyte is E. coli and the ASA is an E. coli specific aptamer (ESA).

In some embodiments of the present disclosure the sensor includes various components including a photoluminescent matrix-material (PMM) that is conjugated with the ASA so that when the ASA binds the analyte, the PMM is configured to respond by altering one or more of its photoluminescent properties. In some embodiments of the present disclosure the photoluminescent emitting properties of the PMM include fluorescent emissions.

In some embodiments of the present disclosure the ASA is a modified ASA that has photoluminescent properties that are altered upon a binding event between the ASA and a food-borne analyte of interest, for example gluten or a gluten constituent-protein. This embodiment may not require a PMM.

In some embodiments of the present disclosure the sensor comprises a modified ASA that is conjugated with a PMM so that the sensor's photoluminescent properties are altered when the modified ASA binds the food-borne analyte of interest, for example gluten or a gluten constituent-protein.

In some embodiments of the present disclosure the sensor comprises a modified ASA that is conjugated with a PMM so that the sensor's photoluminescent properties are altered when the modified ESA binds the food-borne pathogens of interest, for example different strains of E. coli.

In some embodiments of the present disclosure the sensor comprises a modified PMM that is covalently immobilized with blocking agents so that its affinity toward the analyte is suppressed and consequently the GSA can bind with the analyte more efficiently to reduce or substantially prevent a false negative-signal.

In some embodiments of the present disclosure the sensor comprises polyethylene glycol (PEG) as a blocking agent for the PMM.

In some embodiments of the present disclosure the sensor encompasses a chemical modification that includes a modified PMM to reduce or substantially remove the presence of oxygenated functional-groups, which may reduce the oxygenated functional-groups' affinity toward gliadin. Consequently, the GSA can bind with the analyte more efficiently to reduce or substantially prevent a false negative-signal.

Some embodiments of the present disclosure relates to a “turn-on” fluorescent sensor for detecting gluten or a gluten constituent-protein, the interference of other food proteins in gluten free food can also result in fluorescence enhancement resulting in a false positive-signal. To avoid a false positive-signal, the relative decrease in affinity of interfering food proteins towards the ASA may be an important prerequisite. Without loss of generality, in the present embodiments, the sensor comprises a quencher-labelled gliadin (Q-glia) as a competitive agent so that in the gluten-free food. Instead of other food proteins, Q-glia can bind with the GSA to shift the fluorescence resonance energy transfer (FRET) from ‘FAM to PMM’ to ‘FAM to Quencher’ by keeping the emission off and thus minimizing the risk of false positive due to interference of other food proteins.

In some embodiments of the present disclosure BHQ-1 is used as quencher in Q-gliadin conjugate.

### Definitions

Unless defined otherwise, all technical and scientific terms used herein have the same meaning as commonly understood by one of ordinary skill in the art to which this disclosure belongs.

As used herein, the term “about” refers to an approximate +/−10% variation from a given value. It is to be understood that such a variation is always included in any given value provided herein, whether or not it is specifically referred to.

As used herein, the term “analyte” refers to a compound such as a protein, carbohydrate, lipid or combinations thereof that can interact with the sensor embodiments of the present disclosure in a binding event, wherein the binding event changes one or more photoluminescent properties of the sensor. The analyte may be one or more of a gastrointestinal irritant, a contaminant, an allergen or a pathogen or any other type of chemical that can participate in a binding event.

As used herein, the terms “conjugate”, “conjugated” and “conjugating” may be used as a verb to refer to a process where by at least two chemical compounds are linked together to form a “conjugate” (noun) or a “conjugated composition”. For the purposes of this disclosure, the link formed can be any type of chemical bond or interaction between the at least two chemical compounds.

As used here, the term “food-borne analyte” refers to an analyte that may be found within a food substance, either solid or liquid, and then the analyte may be present within a subject that consumes the food substance.

As used herein, the term “gluten” refers to any type of gluten and any gluten constituent-protein including but not limited to gliadin, glutenin, any peptide fragments that are recognizable as being derived from gluten or a gluten constituent-protein, or combinations thereof.

As used herein, the term “photoluminescence” refers to a property of a matter to emit photons after absorbing photons. The emitted photons may have the same energy as the absorbed photons, or not. Photoluminescence is intended to include, but not be limited to, fluorescent emissions of photons.

As used herein, the term ‘Q-glia’ refers to BHQ-gliadin conjugate where BHQ (black hole quencher) is covalently conjugated to gliadin.

In some embodiments of the present disclosure the GSA may be modified to enhance or otherwise alter the photoluminescence properties of the GSA. In some embodiments of the present disclosure the GSA may be modified by attaching a 3′ modifier, a 5′ modifier or a modifier that is located at another position within the GSA sequence. Such modifiers can comprise additional functional groups that may enhance the ASAs' affinity for the analyte or another molecule to facilitate sensing of the GSA and analyte interaction. For example, one embodiment of the present invention relates to a GSA that is modified by the addition of a 6-carboxy fluorescein (6-FAM) at the 3′ end.

Optionally, the GSA may be conjugated with a modified PMM. The PMM may be a nano-scaled PMM. The PMM may be a fluorescent material that is in the nano-scale, or not. Examples of the PMM include, but are not limited to: quantum dots, graphene oxide (GO) nanoparticles, GO nanosheets, GO nanotubes, Buckminster fullerenes, metal nanoparticles (for example gold nanoparticles) or combinations thereof. The PMM and the modifications may work independently of each other or they may work together to provide an additive or synergistically modulated response to a binding event between an analyte of interest and the GSA.

Conjugating the GSA and GO, which may also be referred to as a conjugate or a conjugate-complex, may provide a sensor for detecting gluten. The detection method is based on a change in the photoluminescence properties of the GSA due to the interaction of gluten.

### EXAMPLES

Gli4

One embodiment of the present disclosure relates to a GSA that is referred to herein as Gli4. Gli4 is an GSA of single-stranded deoxyribonucleic acid (ssDNA) that is gliadin specific, with the following nucleic acid sequence (SEQ ID No. 1):

SEQ ID No. 1:
CCAGTCTCCCGTTTACCGCGCCTACACATGTCTGAATGCC.

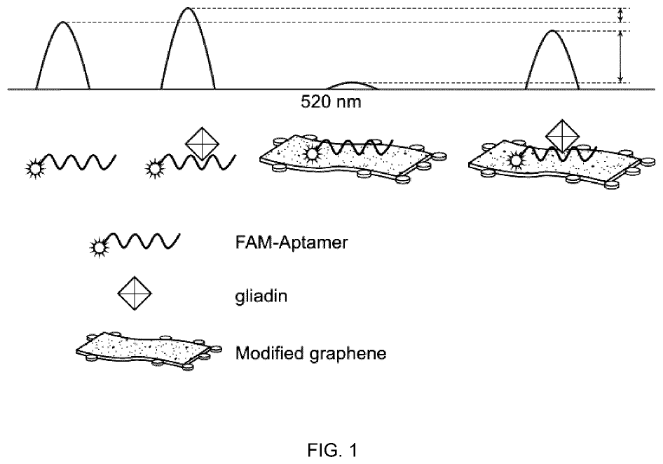

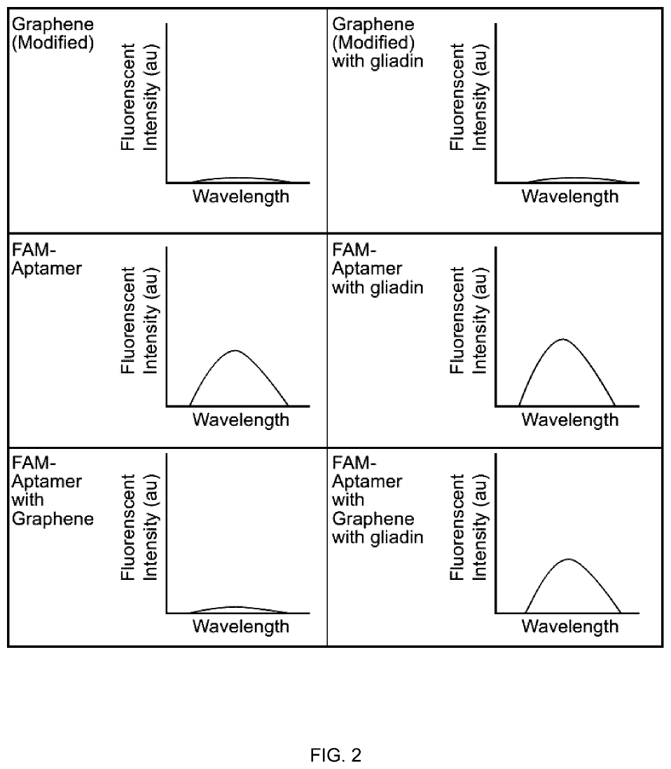

This Gli4 embodiment can detect gluten via quenching of the fluorescence of the GSA-GO conjugate. Reduced PEGylated GO sheets are water dispersible and conjugated with the fluorophore labelled Gli4 GSA. This embodiment may be referred to as the Gli4-FAM-PEG-rGO/Q-glia embodiment. FIG. 1 provides a schematic of the Gli4-FAM-PEG-rGO/Q-glia embodiment. FIG. 2 shows an example of expected trends of fluorescence intensity vs. wavelength data that would be obtained from embodiments of the present disclosure.

Modification of GO was carried out by covalently attaching PEG chains to carboxylic groups on the edges of GO sheets followed by chemical reduction. A suitable amount of GO was dispersed in DMSO through ultrasonication. PEG was added to this dispersion, followed by the addition of dimethylaminopyridine (DMAP) and 1-ethyl-3-(3-dimethylaminopropyl)-carbodiimide (EDC) in inert atmosphere. The reaction mixture was stirred at room temperature for overnight and later centrifuged and washed with water to decant off unreacted PEG.

PEG conjugated GO was then chemically reduced using hydrazine hydrate in aqueous media at about 100° C. The reaction was then centrifuged to wash off unreacted hydrazine to give reduced PEG-GO.

Immobilization of the GO with Gli4 aptamer was carried out and absorption of Gli4 onto the GO was monitored. A suitable amount of GO suspension in tris-EDTA (TE) buffer was ultrasonicated for about 1 hour. A calculated amount of Gli4 was added into the dispersion and the reaction mixture was vortexed for overnight. The excessive and free Gli4 was removed by centrifugation and washing and the mixture was then re-dispersed into TE solution to obtain a suitable concentration of the Gli4-FAM-PEG-rGO/Q-glia conjugate.

The competitive agent Q-glia was synthesized by adding dimethylaminopyridine (DMAP) and 1-ethyl-3-(3-dimethylaminopropyl)-carbodiimide (EDC) to the mixed solution of gliadin and BHQ-1-carboxylic acid in DMSO under inert atmosphere. The reaction mixture was then centrifuged and washed to decant off the unreacted precursors and DMSO to produce a Q-glia conjugate.

Some examples of stock solutions that were used in preparing examples of the present disclosure including:
about 100 μM Gli4-FAM (about pH 8 in 1×TE solution): Tris (about 10 mM), EDTA (about 0.1 mM).
about 2 mg/ml GO Nano colloids (dispersion in water).
1×PBS (Phosphate-buffered saline) (about pH 7.1): Na₂HPO₄ (about 10 mM), KH₂PO₄ (about 1.8 mM), KCl (about 2.7 mM), NaCl (about 137 mM).
about 35 ppm gliadin dissolved in about 70% ethanol/H₂O (three samples of gliadin in PBS prepared and concentrations confirmed through Bradford assay).
10 ppm Q-gliadin dissolved in about 70% ethanol/H₂O (three samples of gliadin in PBS prepared and concentrations confirmed through Bradford assay).

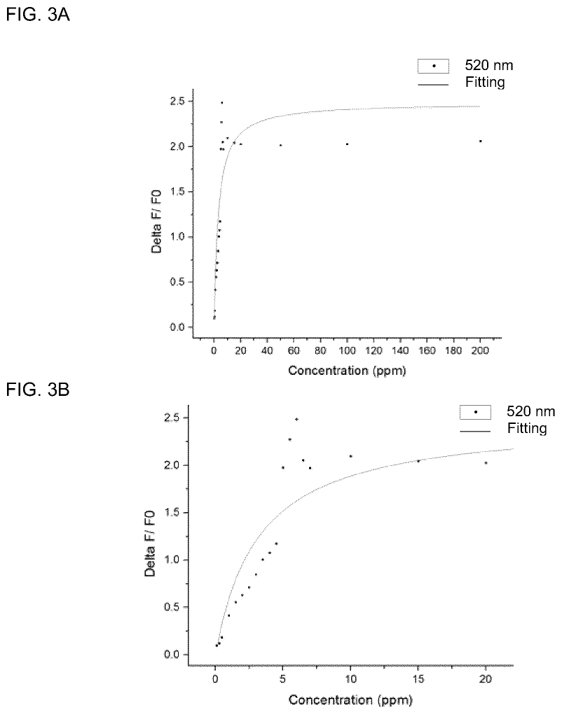

The curve was fitted at 520 nm for Gli4-GO using concentrations of gliadin of between about 0 ppm to about 200 ppm, as shown in FIG. 3A. The curve fitting at 520 nm for concentrations of gliadin between about 0 to about 20 ppm is shown in FIG. 3B where the equation (Eq. 1) for the fitting analysis used was:

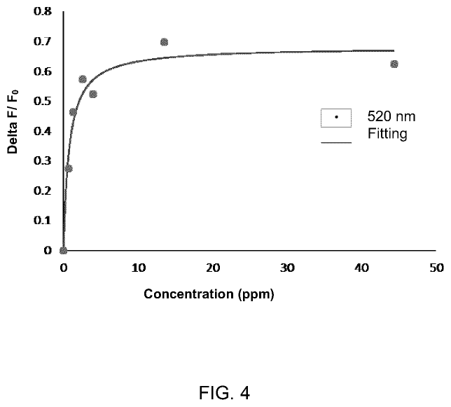

ΔF/Fₒ×100=((ΔFₘₐₓ/Fₒ×100)[L]/(K_{d}+[L]) (Eq. 1)

where ΔFₘₐₓ is the maximum change in fluorescent intensity, L is the analyte concentration, and K_{d} is the dissociation constant). The modified Gli4-FAM-PEG-rGO/Q-glia embodiment showed weak florescent emissions in the absence of gluten. In the presence of gluten the Gli4-FAM-PEG-rGO/Q-glia embodiment demonstrated significantly different fluorescent emitting properties. During a binding experiment with the Gli4-FAM-PEG-rGO/Q-glia embodiments and gliadin, the concentration of Gli4-FAM-PEG-rGO/Q-glia embodiment was about 100 nM and the concentration of gliadin in the solution was increased from about 0 ppm to about 200 ppm. The change in fluorescent emitting properties (change in fluorescence, ΔF, from the initial intensity, F0; ΔF/Fₒ) of the Gli4-FAM-PEG-rGO/Q-glia embodiment over the range of gliadin concentrations is shown in FIG. 4. The relationship between the ratio of fluorescent emissions and gliadin concentration was observed at 520 nm to generate saturation binding of gliadin curves that were fit to logistic algorithms. The parameters and statics for these experimental curves are shown below in Table 1.

TABLE 1
Parameters and statics of curve fitting for the Gli4-FAM-
PEG-rGO/Q-glia embodiment saturation binding of Gliadin.

520 nm

| | Value | Standard Error |
|---|---|---|
| K_{d} | 0.760 ppm | 0.1912 |

| | |
|---|---|
| ΔFₘₐₓ/Fₒ | 0.697 |
| Coefficient of determination | 0.9661 |

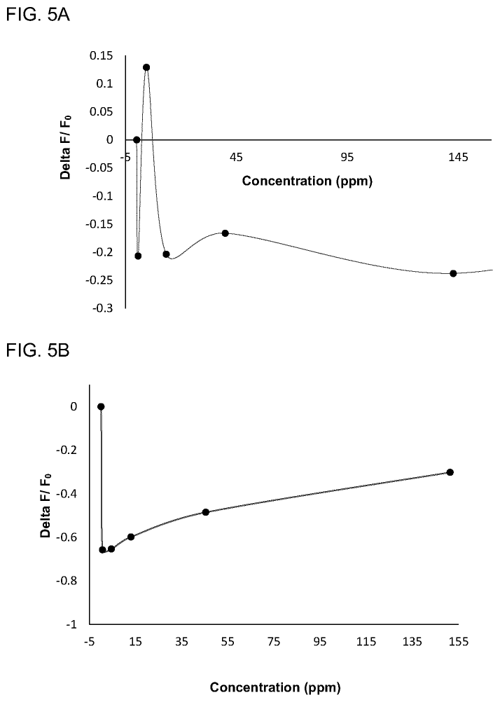

FIG. 5 shows the change in fluorescence emission properties of Gli4-FAM-PEG-rGO/Q-glia embodiment when exposed to lactose protein and peanut proteins. A concentration curve was fit for 520 nm. In these experiments, the concentration of the Gli4-FAM-PEG-rGO/Q-glia embodiment was about 100 nM, whereas the concentrations of lactose and peanut protein ranged from about 0 to about 245 ppm and about 300 ppm, respectively. Both concentration curves were fit according to the equation (Eq. 2):

ΔF/Fₒ×100=(ΔFₘₐₓ/Fₒ×100)[L]/(K_{d}+[L]). (Eq. 2)

The Gli4-FAM-PEG-rGO/Q-glia embodiment shows a dissociation constant (K_{d}) of about 0.11 ppm towards gliadin and a K_{d} of about 13.21 ppm towards lactose.

Binding of the GSA to modified rGO nano-materials may assist the FRET from energy donor fluorophores to energy acceptor GO. The Gli4-FAM-PEG-rGO/Q-glia embodiment may act as a sensor that is sensitive enough to significantly alter its fluorescent emitting properties in the presence of picomolar levels of gluten. This change in fluorescent emitting properties occurs without any complicated detection process, and the Gli4-FAM-PEG-rGO/Q-glia sensor can be made at low cost. Blockers on an oxidized carbon matrix PMM were used followed by a chemical reduction to reduce its affinity to engage in any interaction with gluten proteins and to reduce or substantially prevent false negative-signals that may occur in the absence of gluten. In another embodiment of the present disclosure, a quencher labelled analyte may also or solely be introduced as a competitive conjugate with a higher affinity for the aptamer than other interfering food proteins. This embodiment may also reduce or substantially prevent a false positive-signal that may occur in the absence of gluten. The embodiments of the present disclosure that utilize both false positive-signal preventing approaches may provide a complimentary sensing mechanism for gluten detection with a lower risk of false negative-signals.

Immobilization of the GO with E. coli specific aptamer with the base sequence ID 3 (ESA1-FAM) was carried out and absorption of ESA1-FAM onto the GO was monitored. A suitable amount of GO suspension in tris-EDTA (TE) buffer was ultrasonicated for about 1 hour. A calculated amount of ESA1-FAM was added into the dispersion and the reaction mixture was vortexed for overnight. The excessive and free ESA1-FAM was removed by centrifugation and washing and the mixture was then re-dispersed into TE solution to obtain a suitable concentration of the ESA1-FAM-PEG-rGO conjugate.

GLI4-FAM

In another embodiment of the present disclosure, the GSA has the same nucleic acid sequence as SEQ ID No. 1 but with a 3′ 6-carboxy fluorescein modification (FAM), which is shown as SEQ ID No. 2:

SEQ ID No. 2:
CCAGTCTCCCGTTTACCGCGCCTACACATGTCTGAATGCC-6FAM,

and which may also be referred to herein as a Gli4-FAM embodiment.

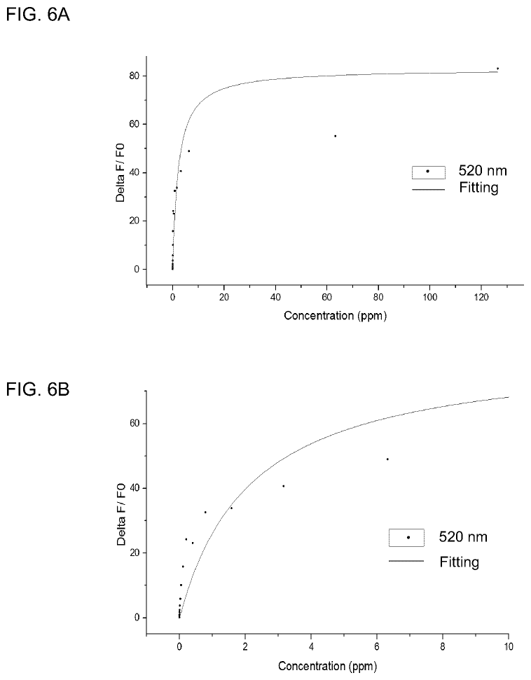

Binding experiments were conducted with the Gli4-FAM embodiment and gliadin. The concentration of Gli4-FAM was about 10 nM and the concentration of gliadin in the solution was increased from about 0.05 nM to about 2×10⁵ nM (about 0.0000316 ppm to about 126.4 ppm). The relationship between the ratio of fluorescence emission and gliadin concentration was explored at 520 nm and 585 nm to generate saturation binding curves, which were then fit to logistic algorithms. The curves that were fitted for the 520 nm observations are shown in FIG. 6A and FIG. 6B. The equation (Eq. 3) used for fitting both wavelengths was:

ΔF/Fₒ×100=(ΔFₘₐₓ/Fₒ×100)[L]/(K_{d}+[L]) (Eq. 3)

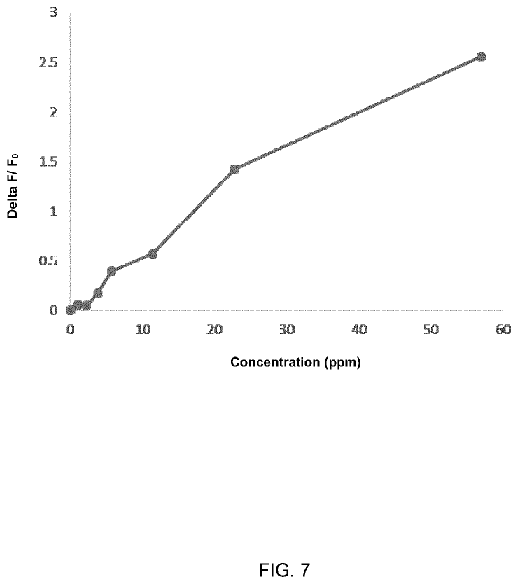

Highly selective but less specific aptamers have a limitation of binding with other food proteins in the absence of the food-borne analyte of interest i.e. gliadin in this case. This challenge can be largely overcome by using much more specific binding units, which is another big challenge in itself. A competitive approach was used to study the binding interactions of Gli4-FAM-PEG-rGO/Q-glia with a competitor Q-gliadin to analyze relative affinity of binding unit of sensing hybrid toward gliadin and quencher labelled gliadin. The hybrid showed much weaker interactions and higher dissociation constant towards Q-gliadin in comparison to toward gliadin itself (see FIG. 7 and Table 2). Without being bound by any particular theory, this result may indicate that any interference caused by Q-gliadin in gluten-rich food may be negligible. However, this result may also exhibit a significant affinity towards sensing the hybrid when there is no gliadin in the absence gluten.

TABLE 2
Parameters and statics of curve fitting for Gli4-
FAM-PEG-rGO saturation binding of Q-gliadin.

520 nm

| | Value | Standard Error |
|---|---|---|
| K_{d} | 148.5 ppm | 0.02 |

| | |
|---|---|
| ΔFₘₐₓ/Fₒ | 3.78 |
| Coefficient of determination | 0.9654 |

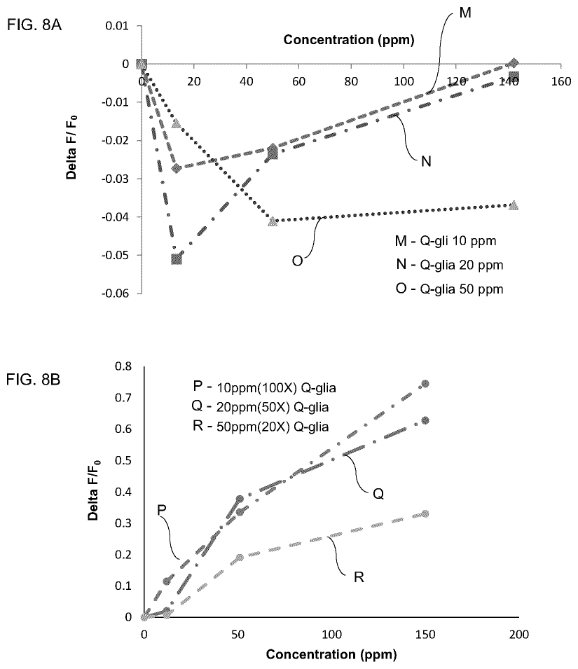

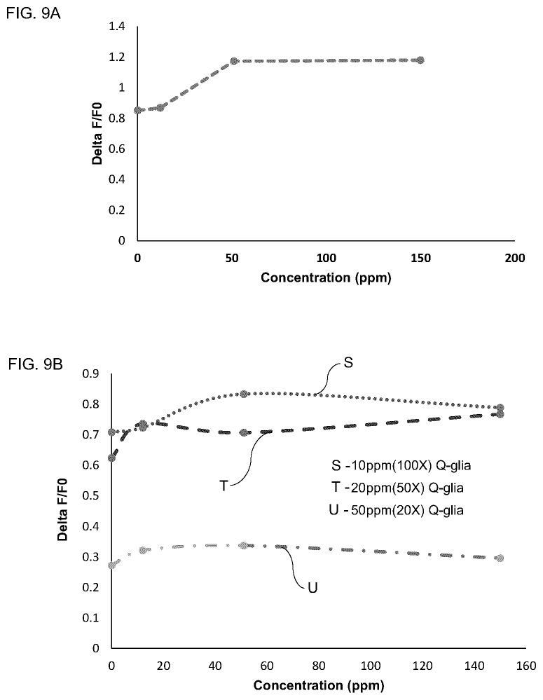

FIG. 8A and FIG. 8B each show an example of an interfering response of lactose and peanut proteins toward fluorescence of competitor Q-glia that incorporated sensing hybrid Gli4-FAM-PEG-rGO. Although both lactose and peanut proteins showed a contrasting response toward the competitive agent, the increase in concentration of Q-glia decreased the relative emission intensity of the hybrid. Since the peanut protein showed a relatively higher-degree of interference toward the competitive agent, the interaction of peanut protein toward the hybrid in the absence and presence of Q-glia was studied with an example of the data obtained shown in FIG. 9A and FIG. 9B. In the absence of a competitor (Q-glia), the peanut protein showed increase in emission intensity with an increase in its concentration in response to a constant concentration of standard gliadin in Gli4-FAM-PEG-rGO hybrid. However, when the concentration of Q-glia was increased from about 10 to about 50 ppm, the overall emission intensity was observed to decrease. The fluorescence intensity of the hybrid in the presence of gliadin with a higher concentration of Q-glia (about 50 ppm) did not exhibit any change with an increase in the concentration of the peanut proteins. Therefore, this example may be beneficial to decrease or substantially eliminate the incidence of false positive-signals, in the absence of gluten, due to interfering proteins.

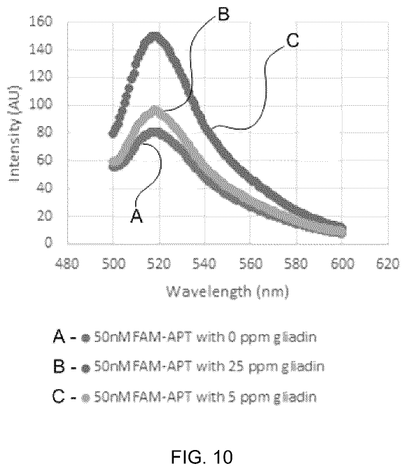

FIG. 10 shows an example of a fluorescence emission detected in the absence of GO. FIG. 10 shows the background fluorescence emitted when a control measurement of the experiment is observed in the presence of the gliadin analyte in the following concentrations: about 0 ppm (A); about 5 ppm (B); and about 25 ppm (C) in about 50 nM Gli4-FAM and the peak attained by the maximum concentration of the gliadin analyte of about 25 ppm in FIG. 10. The control experiment is when the sensor that measures the presence of the analyte is measured in the absence of the analyte (about 0 ppm (A)). This control experiment may provide a reference over which any increase in fluorescence emission may be observed to indicate the presence of the analyte when the Gli4-FAM is highly specific to gliadin. The fluorescence emission characteristics of the control illustrates a high background-signal despite of the absence of the analyte. This observation may provide a reason for integrating the GO into the construction of the sensor: i.e. to drive the control/background/reference to a lower intensity.

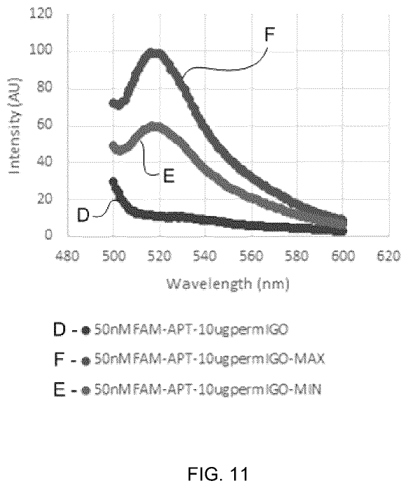

FIG. 11 shows data obtained when GO was used as a quencher to the fluorophore, the GO was attached to the 3′ side of the Gli4 aptamer. In FIG. 11 all data traces were obtained using about 10 μg/ml GO and about 50 nM of Gli4-FAM. Line D represents when there was no gliadin analyte present, line E represents when there was about 5 ppm gliadin analyte (MIN) and line F represents when there was about 25 ppm gliadin analyte (MAX).

The 50 nM Gli4-FAM with 10 μg/ml of GO was prepared as follows:
1. Prepared about 10 μM Gli4-FAM by mixing about 100 μl of Gli4-FAM with about 900 μl of 1×PBS.
2. Added about 6.5 μl of 10 μM Gli4-FAM with about 6.5 μl of about 2 mg/ml GO solution to a total volume of about 1300 μl. The volume was made up by 1×PBS.

After adding GO into the FAM-Gli4, the fluorescence peak was much lower than when GO was not included. For example, when about 10 μg/ml GO was added to about 50 nM of Gli4-FAM, the peaks of the background/reference/control were reduced by about 88%.

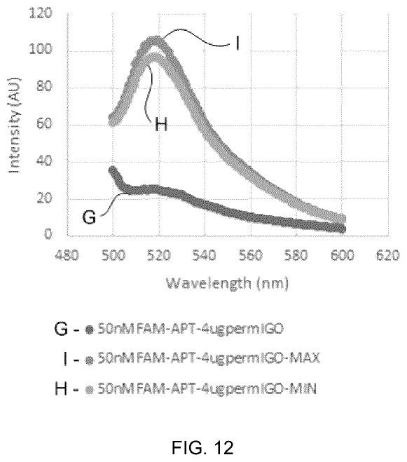

FIG. 12 shows an example of data traces that were obtained with about 4 μg/ml GO and about 50 nM of Gli4-FAM. Line G represents when there was no gliadin analyte present, line H represents when there was about 5 ppm gliadin analyte and line I represents when there was about 25 ppm gliadin analyte. The detection of about 5 ppm gliadin increased by about 188% in comparison to when there was 4 μg/ml GO and about 50 nM of Gli4-FAM (see line B in FIG. 10 vs. line H in FIG. 12).

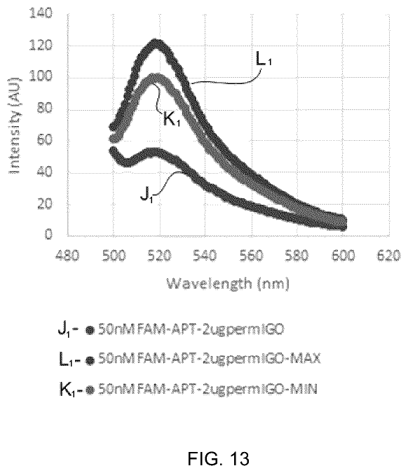

FIG. 13. shows an example of data traces that were obtained with about 2 μg/ml GO and about 50 nM of Gli4-FAM. Line J represents when there was no gliadin analyte present, line K represents when there was about 5 ppm gliadin analyte and line L represents when there was about 25 ppm gliadin analyte.

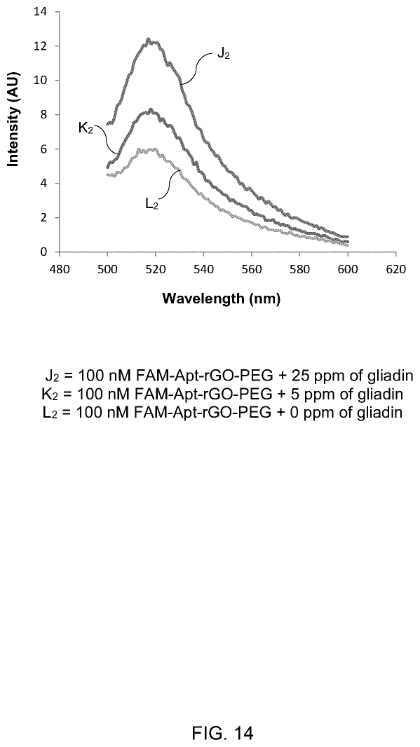

FIG. 14 shows an example of data traces that exhibit the interactions of low and high concentrations of gliadin standards with a sensing hybrid that includes about 100 nM of the FAM-Aptamer immobilized over a modified reduced graphene oxide (rGO). An increase in the emission intensity was observed with an increase in the concentration of the analyte. This relationship may show the strong interactions that occur between the sensing hybrid and gliadin.

To obtain the data in FIG. 10 to FIG. 14, the measurements were performed in a spectrometer set at “medium” scan speed, with the excitation set at 490 nm, and the emission range was between 500 nm to 600 nm. Both the excitation and the emission slits were set at 5 nm.

ESA1-FAM

In another embodiment of the present disclosure, the ASA is an ESA with a nucleic acid sequence and a 3′ 6-carboxy fluorescein modification (FAM), which is shown as SEQ ID No. 3:

SEQ ID No. 3:
TCGTGCAGCAGGGGCTGTGTCGCGGTCGGTAGTGCTGTGGTGCG-6FAM

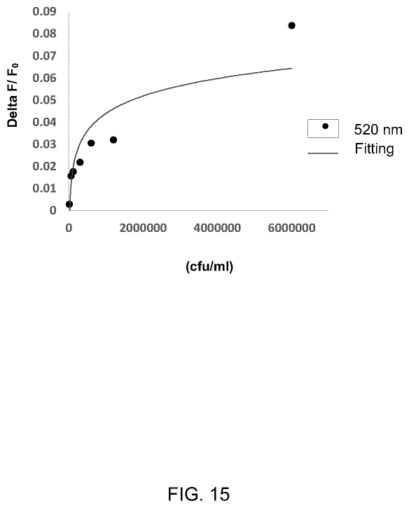

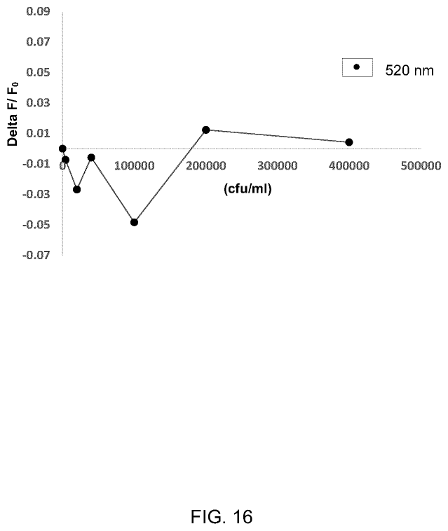

Binding experiments were conducted with the ESA-PEG-rGO embodiment and E. coli O157. The concentration of ESA1-FAM-PEG-rGO was about 100 nM and the optical density of maximum concentration of E. coli in the solution was 0.5 a.u., which was subsequently diluted further up to 10⁴ folds. The relationship between the ratio of fluorescence emission and E. coli concentration was explored at 520 nm to generate saturation binding curves, which were then fit to logistic algorithms. The curves that were fitted for the 520 nm observations are shown in FIG. 15 and FIG. 16. FIG. 15 shows a standard binding curve of 100 nM of ESA in ESA1-PEG-rGO in the presence of varying amount of colony forming units per ml of E. coli O157. FIG. 16 shows weak standard binding curve of 100 nM of ESA in ESA1-FAM-PEG-rGO in the presence of varying amount of colony forming units per ml of E. coli ATCC 25922. This non-significant binding behavior of ESA1-FAM-PEG-rGO indicates its selective affinity towards the target analyte E. coli O157.

In other embodiments of the present disclosure there may be other variations of the absolute and relative concentration of the components of the sensor. For example, the amount of GO to be used in conjunction with the ASA can vary from about 1 μg/ml to about 2 mg/ml and various concentrations of ASA can be used. Additionally, the concentration of covalently conjugated blocking agents used can depend on the number of functional groups on the PMM undergoing a substitution reaction.

1. A sensor for detecting a food-borne analyte, the sensor comprising a photoluminescent matrix-material (PMM) and an analyte-specific aptamer (ASA), wherein the PMM is configured to change a photoluminescent property when a binding event occurs between the sensor and the food-borne analyte.

2. The sensor of claim 1, wherein the PMM is modified to block interfering functional groups of the PMM.

3. The sensor of claim 2, wherein the blocked PMM is chemically reduced to remove or substantially remove attached interfering functional-groups.

4. The sensor of claim 3, wherein the reduced and blocked PMM is conjugated with the ASA.

5. The sensor of claim 1, wherein the ASA is modified to provide a photoluminescent property to the ASA.

6. The sensor of claim 5, wherein the photoluminescent property is provided by attaching a photoluminescent molecule to the ASA.

7. The sensor of claim 6, wherein the photoluminescent molecule is a fluorophore that is attached to the ASA.

8. The sensor of claim 1, wherein the photoluminescent property comprises emitting photons.

9. The sensor of claim 1, wherein the photoluminescent property comprises emitting fluorescence.

10. The sensor of claim 1, wherein the ASA is configured to recognize one or more of a gastrointestinal irritant, a gastrointestinal contaminant, a gastrointestinal allergen, a gastrointestinal pathogen or combinations thereof.

11. The sensor of claim 10, wherein the ASA is configured to recognize gluten.

12. The sensor of claim 11, wherein gluten comprises a gluten constituent-protein, any peptide fragments that are recognizable as being derived from gluten or combinations thereof.

13. The sensor of claim 12, wherein the gluten constituent-protein is one or more of gliadin, glutenin or combinations thereof.

14. The sensor of claim 1, wherein the ASA comprises SEQ ID No. 1.

15. The sensor of claim 1, wherein the ASA comprises SEQ ID No. 2.

16. The sensor of claim 1, wherein the ASA is configured to recognize a strain of bacteria.

17. The sensor of claim 15, wherein the bacteria is E. coli.

18. The sensor of claim 16, wherein the ASA comprises SEQ ID No. 3.

19. The sensor of claim 1, wherein a quencher-analyte conjugate is incorporated into a hybrid system and the hybrid system is configured to reduce or substantially prevent a false positive-signal.


SEQUENCE LISTING (3 sequences)

SEQ ID NO: 1 - DNA, 40 bp, Artificial Sequence
  Synthetic Sequence
ccagtctcccgtttaccgcgcctacacatgtctgaatgcc

SEQ ID NO: 2 - DNA, 40 bp, Artificial Sequence; identical to SEQ ID NO: 1
  Synthetic Sequence
  misc_feature (40)..(40): 3′ 6-carboxy fluorescein modification (FAM)

SEQ ID NO: 3 - DNA, 44 bp, Artificial Sequence
  Synthetic Sequence
  misc_feature (44)..(44): 3′ 6-carboxy fluorescein modification (FAM)
tcgtgcagcaggggctgtgtcgcggtcggtagtgctgtggtgcg